(19) United States
(12) Patent Application Publication
(10) Pub. No.: US 2025/0209636 A1
(43) Pub. Date: Jun. 26, 2025

(54) MEDICAL IMAGING SYSTEM AND METHOD FOR GENERATING SCAN RANGE INDICATOR IN MEDICAL IMAGING SYSTEM

(71) Applicant: GE Precision Healthcare LLC, Waukesha, WI (US)

(72) Inventors: Xinyuan Wang, Beijing (CN); Ziyu Liu, Beijing (CN); Feng Li, Beijing (CN)

(21) Appl. No.: 18/988,828

(22) Filed: Dec. 19, 2024

(30) Foreign Application Priority Data
Dec. 25, 2023 (CN) 202311797503.1

(51) Int. Cl. G06T 7/149; G06T 7/593

(52) U.S. Cl. CPC G06T 7/149; G06T 7/596; G06T 2207/10028

(57) ABSTRACT

Provided are a medical imaging system and a method for generating a scan range indicator in the medical imaging system. The method includes projecting a 3D point cloud, generated based on a depth image of a patient, onto a plane perpendicular to a table supporting the patient and along a scanning direction, so as to generate a projected 2D point cloud; generating a patient 2D point cloud contour based on the projected 2D point cloud; based on an upper and lower positions that are received in real time and indicate a scan range, determining two corresponding points on the patient 2D point cloud contour, the upper and lower boundary positions; and, using the scan positions of the corresponding points as base points, presenting an upper boundary line and a lower boundary line of the scan range on an image of the patient as a scan range indicator.

### CROSS-REFERENCE TO RELATED APPLICATIONS

This application claims priority to Chinese Application No. 202311797503.1, filed on Dec. 25, 2023, the disclosure of which is incorporated herein by reference in its entirety.

### TECHNICAL FIELD

The present disclosure relates to non-invasive diagnostic imaging, and more particularly, relates to a medical imaging system and a method for generating a scan range indicator in the medical imaging system.

### BACKGROUND

In conventional medical imaging scans, an operator needs to first instruct a patient to lie down on a scanning table, and then manually move the scanning table and use a laser light in a scanning gantry to determine baselines and landmarks for scanning. The scanning range is determined by an offset, while the offset is determined by parameter values that need to be set on a console outside a scanning room. This approach does not provide an intuitive way for the operator to understand the scanning range and requires the operator to operate both inside and outside the scanning room.

Some techniques have been developed in an attempt to render a two-dimensional (2D) image on an image of a patient, to display a scan range. However, such techniques have parallax problems, particularly in the head region, such that it is still difficult for the operator to accurately identify the scanning volume of the patient from the image.

### SUMMARY

The object of the present disclosure is to overcome the above and/or other problems in the prior art, so that a scan range indicator can be visually presented, and said parallax problems can be ameliorated.

According to a first aspect of the present disclosure, a method for generating a scan range indicator in a medical imaging system is provided, including: projecting a 3D point cloud, generated on the basis of a depth image of a patient, onto a plane perpendicular to a scanning table supporting the patient and along a scanning direction, so as to generate a projected 2D point cloud; generating a patient 2D point cloud contour on the basis of the projected 2D point cloud; on the basis of an upper boundary position and a lower boundary position that are received in real time and indicate a scan range, determining two corresponding points on the patient 2D point cloud contour, the upper boundary position and the lower boundary position indicating scan positions in the scanning direction; and, using the scan positions of the corresponding points as base points, presenting an upper boundary line and a lower boundary line of the scan range on an image of the patient as a scan range indicator.

In one embodiment, the upper boundary line and the lower boundary line may have a linear shape. In one embodiment, a midpoint of the upper boundary line may coincide with one of the corresponding points, and a midpoint of the lower boundary line may coincide with the other of the corresponding points. In one embodiment, the patient 2D point cloud contour can be generated by selecting a highest point at each scan position as a contour height.

In one embodiment, the method may further include: dividing the projected 2D point cloud of the patient into a head region and a torso region on the basis of position information of the scanning table; identifying a highest point in the head region; and replacing, with the height of the highest point, the heights of all points on the patient 2D point cloud contour that are located at one side of the top of the patient's head relative to the highest point.

In one embodiment, the position information of the scanning table may comprise position information of a support for placing the patient's head. In one embodiment, determining the two corresponding points on the patient 2D point cloud contour may include determining points on the patient 2D point cloud contour that are closest to the upper boundary position and the lower boundary position, respectively, to be the corresponding points. In one embodiment, determining the two corresponding points on the patient 2D point cloud contour may include interpolating nearest points on both sides of the upper boundary position and the lower boundary position, respectively, to be the corresponding points. In one embodiment, the depth image of the patient may be obtained by a depth camera which may be fixedly mounted in the medical imaging system.

According to a second aspect of the present disclosure, a method for generating a scan range indicator in a medical imaging system is provided. The method includes: projecting a 3D point cloud, generated on the basis of a depth image of a patient, onto a plane perpendicular to a scanning table supporting the patient and along a scanning direction, so as to generate a projected 2D point cloud; on the basis of an upper boundary position and a lower boundary position that are received in real time and indicate a scan range, determining corresponding points in the projected 2D point cloud, the upper boundary position and the lower boundary position indicating scan positions in the scanning direction, and the corresponding points being highest points at the corresponding scan positions; and, using the scan positions of the corresponding points as base points, presenting an upper boundary line and a lower boundary line of the scan range on an image of the patient as a scan range indicator.

In one embodiment, the upper boundary line and the lower boundary line may have a linear shape. In one embodiment, a midpoint of the upper boundary line may coincide with one of the corresponding points, and a midpoint of the lower boundary line may coincide with the other of the corresponding points. In one embodiment, the method may further include: dividing the projected 2D point cloud of the patient into a head region and a torso region on the basis of position information of the scanning table; identifying a highest point in the head region; and replacing, with the height of the highest point, the heights of all points in the patient 2D point cloud that are located at one side of the top of the patient's head relative to the highest point.

In one embodiment, the position information of the scanning table may include position information of a support for placing the patient's head. In one embodiment, determining the corresponding points in the projected 2D point cloud may include determining highest points at the scan positions closest to the upper boundary position and the lower boundary position on the patient 2D point cloud contour, respectively, to be the corresponding points. In one embodiment, determining the corresponding points in the projected 2D point cloud may include: interpolating highest points at the scan positions on both sides of the upper boundary position and the lower boundary position, respectively, to be the corresponding points.

In one embodiment, the depth image of the patient may be obtained by a depth camera which may be fixedly mounted in the medical imaging system. According to a third aspect of the present disclosure, a medical imaging system is provided, including: a depth camera configured to acquire a depth image of a patient; a scanning table configured to support the patient; a scan planning apparatus configured to receive an upper boundary position and a lower boundary position that indicate a scan range; a display configured to display an image of the patient; and a processor. The processor is configured to: project a 3D point cloud, generated on the basis of the depth image of the patient acquired by the depth camera, onto a plane perpendicular to the scanning table and along a scanning direction, so as to generate a projected 2D point cloud; generate a patient 2D point cloud contour on the basis of the projected 2D point cloud; on the basis of the upper boundary position and the lower boundary position that are received in real time and indicate the scan range, determine corresponding points on the patient 2D point cloud contour, the upper boundary position and the lower boundary position indicating scan positions in the scanning direction; and, using the scan positions of the corresponding points as base points, cause the display to present an upper boundary line and a lower boundary line of the scan range on an image of the patient.

According to a fourth aspect of the present disclosure, a medical imaging system is provided, including: a depth camera configured to acquire a depth image of a patient; a scanning table configured to support the patient; a scan planning apparatus configured to receive an upper boundary position and a lower boundary position that indicate a scan range; a display configured to display an image of the patient; and a processor. The processor is configured to: project a 3D point cloud, generated on the basis of the depth image of the patient acquired by the depth camera, onto a plane perpendicular to the scanning table and along a scanning direction, so as to generate a projected 2D point cloud; on the basis of the upper boundary position and the lower boundary position that are received in real time and indicate the scan range, determine corresponding points in the projected 2D point cloud, the upper boundary position and the lower boundary position indicating scan positions in the scanning direction, and the corresponding points being highest points at corresponding scan positions thereof; and, using the scan positions of the corresponding points as base points, cause the display to present an upper boundary line and a lower boundary line of the scan range on an image of the patient.

According to a fifth aspect of the present disclosure, a computer-readable medium having instructions stored thereon is provided, the instructions, when executed by a processor, causing the processor to perform the above method.

### BRIEF DESCRIPTION OF THE DRAWINGS

The present disclosure can be better understood by means of the description of the exemplary embodiments of the present disclosure in conjunction with the drawings, in which:

FIG. 1 shows an exemplary CT imaging system 100;

FIG. 2 shows an exemplary imaging system 200 similar to the CT imaging system 100 in FIG. 3;

FIG. 3 illustrates a logic diagram of generating a scan range indicator according to a technique of the present disclosure;

FIG. 4(A) shows an example original 3D point cloud generated from a depth image 13;

FIG. 4(B) shows an example projected 2D point cloud generated from the original 3D point cloud;

FIG. 4(C) shows an example projected 2D point cloud contour generated from the projected 2D point cloud;

FIG. 4(D) shows an example projected 2D point cloud contour subjected to filling processing;

FIG. 5 illustrates a schematic diagram of generating a scan range indicator according to a technique of the present disclosure;

FIG. 6 illustrates a flow diagram of an example generation method 600 of a scan range indicator according to a technique of the present disclosure;

FIG. 7 illustrates a flow diagram of another example generation method 700 of a scan range indicator according to a technique of the present disclosure;

FIG. 8 illustrates a block diagram of an exemplary imaging system 800 according to a technique of the present disclosure; and

FIG. 9 illustrates an exemplary block diagram of a computing device 900 according to a technique of the present disclosure.

In the accompanying drawings, similar components and/or features may have the same numerical reference signs. Further, components of the same type may be distinguished by letters following the reference sign, and the letters may be used for distinguishing between similar components and/or features. If only a first numerical reference sign is used in the specification, the description is applicable to any similar component and/or feature having the same first numerical reference sign irrespective of the subscript of the letter.

### DETAILED DESCRIPTION

Specific embodiments of the present disclosure will be described below, but it should be noted that in the specific description of these embodiments, for the sake of brevity of description, it is impossible to describe all features of the actual embodiments of the present disclosure in detail in this description. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for a person of ordinary skill in the art related to the content disclosed in the present disclosure, some design, manufacture, or production changes made on the basis of the technical content disclosed in the present disclosure are only common technical means, and should not be construed as the content of the present disclosure being insufficient.

References in the specification to “an embodiment,” “embodiment,” “exemplary embodiment,” and so on indicate that the embodiment described may include a specific feature, structure, or characteristic, but the specific feature, structure, or characteristic is not necessarily included in every embodiment. Besides, such phrases do not necessarily refer to the same embodiment. Further, when a specific feature, structure, or characteristic is described in connection with an embodiment, it is believed that affecting such feature, structure, or characteristic in connection with other embodiments (whether or not explicitly described) is within the knowledge of those skilled in the art.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

Unless otherwise defined, the technical or scientific terms used in the claims and the description should be as they are usually understood by those possessing ordinary skill in the technical field to which they belong. The terms “include” or “comprise” and similar words indicate that an element or object preceding the terms “include” or “comprise” encompasses elements or objects and equivalent elements thereof listed after the terms “include” or “comprise”, and do not exclude other elements or objects.

Currently, some techniques have been developed for intuitively rendering and presenting scan range boundaries for medical imaging scanning of a patient on a screen viewed by an operator. For example, a curved contour line of a patient surface is acquired by a depth camera, and the curved contour line is presented according to operator configuration of the scan boundary. Since the depth camera is usually mounted on the ceiling of a scanning room or at an upper part of a medical imaging device, the field of view of the depth camera covers a scanning table and a patient carried on the scanning table. The depth camera presents a different angle with respect to the scanning table or the patient carried on the scanning table at each medical imaging scan position along an axial or longitudinal direction, and the patient has a certain thickness, so a scan range contour line acquired by the depth camera may have a parallax problem.

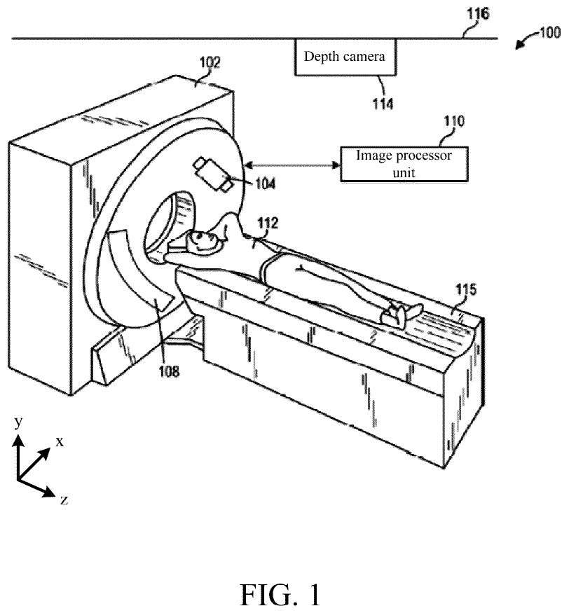

To solve the described problem, the present disclosure provides a method for generating a scan range indicator on the basis of a depth image of a patient and a medical imaging system using the same, which can visually and intuitively present a boundary of a scan range in an image of the patient, while alleviating parallax problems due to an opposite orientation of the patient and a depth camera, thereby improving consistency of the scan range indicator with an actual scan range. Embodiments of the present disclosure will be described below by way of example with reference to FIG. 1 to FIG. 9. The following description relates to various examples of the imaging system.

While a CT system is described by way of example, it should be understood that the techniques of the present disclosure may also be useful when applied to images acquired by using other imaging modalities, such as an X-ray imaging system, a magnetic resonance imaging (MRI) system, a positron emission tomography (PET) imaging system, a single photon emission computed tomography (SPECT) imaging system, and combinations thereof (e.g., a multi-modal imaging system such as a PET/CT, PET/MR, or SPECT/CT imaging system). The discussion of the CT imaging system in the present disclosure is provided only as an example of one suitable imaging system.

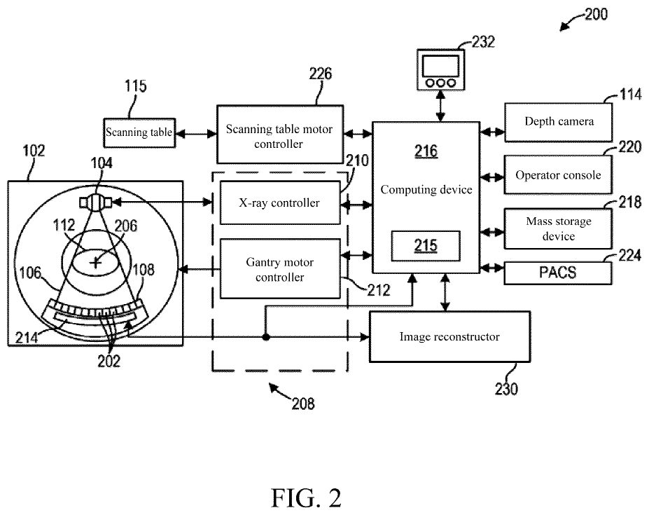

FIG. 1 shows an exemplary CT imaging system 100. Specifically, the CT imaging system (also referred to as a CT device) 100 is configured to image a subject 112 (such as a patient, an inanimate subject, one or more manufactured components, an industrial component, a foreign subject, or the like). Throughout the present disclosure, the terms “subject”, “scan subject” and “examination subject” may be used interchangeably, and it should be understood that, at least in some embodiments, a patient is a type of subject that may be imaged by the CT imaging system 100, and that a subject may include a patient. In some embodiments, the CT imaging system 100 includes a gantry 102, which may include at least one X-ray radiation source 104. The at least one X-ray radiation source 104 is configured to project an X-ray beam (or X-ray) 106 (see FIG. 2) for imaging the subject 112. Specifically, the X-ray radiation source 104 is configured to project the X-ray 106 toward a detector array 108 positioned on the opposite side of the gantry 102. Although FIG. 1 illustrates only one X-ray radiation source 104, in some embodiments, a plurality of X-ray radiation sources 104 may be used to project a plurality of X-rays 106 toward a plurality of detectors, so as to acquire projection data corresponding to the subject 112 at different energy levels.

In some embodiments, the X-ray radiation source 104 projects a fan-shaped or cone-shaped X-ray beam 106. The fan-shaped or cone-shaped X-ray beam 106 is collimated to be located in an x-y plane of a Cartesian coordinate system, and the plane is generally referred to as an “imaging plane” or a “scanning plane”. The X-ray beam 106 passes through the subject 112. The X-ray beam 106, after being attenuated by the subject 112, is incident on the detector array 108. The intensity of the attenuated radiation beam received at the detector array 108 depends on the attenuation of the X-ray 106 by the subject 112. Each detector element of the detector array 108 produces a separate electrical signal that serves as a measure of the intensity of the beam at the detector position. Intensity measurements from all detectors are separately acquired to generate a transmission distribution.

In third-generation CT imaging systems, the gantry 102 is used to rotate the X-ray radiation source 104 and the detector array 108 within the imaging plane around the subject 112, so that the angle at which the X-ray beam 106 intersects with the subject 112 is constantly changing. A full gantry rotation occurs when the gantry 102 completes a full 360-degree rotation. A set of X-ray attenuation measurements (e.g., projection data) from the detector array 108 at one gantry angle is referred to as a “view”. Thus, the view represents each incremental position of the gantry 102. A “scan” of the subject 112 includes a set of views made at different gantry angles or viewing angles during one rotation of the X-ray radiation source 104 and the detector array 108.

In some examples, the CT imaging system 100 may include a depth camera 114 positioned on or outside the gantry 102. As shown in FIG. 1, the depth camera 114 is mounted on a ceiling panel 116 positioned above the subject 112 and oriented to image the subject when the subject 112 is at least partially outside the gantry 102. The depth camera 114 may include one or more light sensors, including one or more visible light sensors and/or one or more infrared (IR) light sensors. In some embodiments, the one or more IR sensors may include one or more sensors in a near-IR range and a far-IR range to implement thermal imaging. In some embodiments, the depth camera 114 may further include an IR light source. The light sensor may be any 3D depth sensor, such as a time-of-flight (ToF) sensor, a stereo sensor, or a structured light depth sensor, the 3D depth sensor being operable to generate a 3D depth image, while in other embodiments the light sensor may be a two-dimensional (2D) sensor operable to generate a 2D image. In some such embodiments, a 2D light sensor may be used to infer a depth from knowledge of light reflection to estimate a 3D depth. Regardless of whether the light sensor is a 3D depth sensor or a 2D sensor, the depth camera 114 may be configured to output a signal encoding an image to a suitable interface. The interface may be configured to receive, from the depth camera 114, the signal encoding the image. A 3D point cloud of an object within the field of view of the depth camera 114 may be generated based on the signal for encoding the image, and a 3D point cloud of the subject 112 can be extracted from the 3D point cloud of the object.

The CT imaging system 100 further includes an image processing unit 110 configured to present an image of a patient, to render and present a scan range indicator on the image of the patient using the method described herein, and to reconstruct an image of a target volume of the patient using a suitable reconstruction method (such as an iterative or analytical image reconstruction method).

The CT imaging system 100 further includes a scanning table 115, and the subject 112 is positioned on the scanning table to facilitate imaging. The scanning table 115 may be electrically powered, so that a vertical position and/or a lateral position of the scanning table can be adjusted. Accordingly, the scanning table 115 may include a motor and a motor controller, as will be explained below with respect to FIG. 2. The scanning table motor controller moves the scanning table 115 by adjusting the motor, so as to properly position the subject in the gantry 102 to acquire projection data corresponding to the target volume of the subject. The scanning table motor controller may adjust the height of the scanning table 115 (e.g., a vertical position relative to a floor on which the scanning table is located) and a lateral position of the scanning table 115 (e.g., a horizontal position of the scanning table along an axis parallel to an axis of rotation of the gantry 102).

FIG. 2 shows an exemplary imaging system 200 similar to the CT imaging system 100 in FIG. 1. In some embodiments, the imaging system 200 includes the detector array 108 (see FIG. 1). The detector array 108 further includes a plurality of detector elements 202, which together acquire the X-ray beam 106 (see FIG. 1) passing through the subject 112 to acquire corresponding projection data.

In some embodiments, the imaging system 200 includes a control mechanism 208 to control the movement of the components, such as the rotation of the gantry 102 and the operation of the X-ray radiation source 104. In some embodiments, the control mechanism 208 further includes an X-ray controller 210, the X-ray controller 210 being configured to provide power and timing signals to the X-ray radiation source 104. Additionally, the control mechanism 208 includes a gantry motor controller 212, configured to control the rotational speed and/or position of the gantry 102 on the basis of imaging requirements.

In some embodiments, the control mechanism 208 further includes a data acquisition system (DAS) 214, configured to sample analog data received from the detector elements 202, and convert the analog data to a digital signal for subsequent processing. The data sampled and digitized by the DAS 214 is transmitted to a computer or computing device 216. In an example, the computing device 216 stores data in a storage apparatus 218. For example, the storage apparatus 218 may include a hard disk drive, a floppy disk drive, a compact disc-read/write (CD-R/W) drive, a digital versatile disc (DVD) drive, a flash drive, and/or a solid-state storage drive.

Additionally, the computing device 216 provides commands and parameters to one or more of the DAS 214, the X-ray controller 210, and the gantry motor controller 212 to control system operations, such as data acquisition and/or processing. In some embodiments, the computing device 216 controls system operations on the basis of operator input. The computing device 216 receives the operator input by means of an operator console 220 that is operably coupled to the computing device 216, the operator input including, for example, commands and/or scan parameters. The operator console 220 may include a keyboard (not shown) or a touch screen to allow the operator to specify commands and/or scan parameters.

Although FIG. 2 shows only one operator console 220, more than one operator console may be coupled to the imaging system 200, for example, for inputting or outputting system parameters, requesting examination, and/or viewing images. Moreover, in some embodiments, the imaging system 200 may be coupled to, for example, a plurality of displays, printers, workstations, and/or similar devices located locally or remotely within an institution or hospital or in a completely different location by means of one or more configurable wired and/or wireless networks (such as the Internet and/or a virtual private network).

In some embodiments, for example, the imaging system 200 includes or is coupled to a picture archiving and communication system (PACS) 224. In one exemplary embodiment, the PACS 224 is further coupled to a remote system (such as a radiology information system or a hospital information system), and/or an internal or external network (not shown) to allow operators in different locations to provide commands and parameters and/or acquire access to image data.

The computing device 216 uses operator-provided and/or system-defined commands and parameters to operate the scanning table motor controller 226, the scanning table motor controller being able to control the scanning table motor, thereby adjusting the position of the scanning table 115 shown in FIG. 1. Specifically, the scanning table motor controller 226 moves the scanning table 115 by means of the scanning table motor, so as to properly position the subject 112 in the gantry 102 to acquire projection data corresponding to a target volume of the subject 112. For example, the computing device 216 may send a command to the scanning table motor controller 226, so as to instruct the scanning table motor controller 226 to adjust the vertical position and/or the lateral position of the scanning table 115 by means of the motor.

In some embodiments, the display 232 may allow the operator to select a volume of interest (VOI) and/or request subject information, for example, by means of a graphical user interface (GUI), for subsequent scanning or processing.

As described further herein, the computing device 216 may include computer-readable instructions executable to present, on the basis of a depth image of the subject 112, a range boundary of an axial scan on the image of the subject 112.

The depth camera 114 may be operably and/or communicatively coupled to the computing device 216 to provide image data to determine the anatomy of the subject, including posture and orientation. Additionally, various methods and procedures described further herein for presenting a boundary of the scan range in the image of the patient on the basis of the depth image data generated by the depth camera 114 may be stored as executable instructions in a non-transitory memory of the computing device 216.

Additionally, in some examples, the computing device 216 may include a camera image data processor 215 that includes instructions for processing information received from the depth camera 114. The information (which may include depth information and/or visible light information) received from the depth camera 114 may be processed to determine various parameters of the subject, such as the identity of the subject, the physique of the subject (e.g., height, weight, and patient thickness), and the current position of the subject relative to the scanning table and the depth camera 114. For example, prior to imaging, the body contour or anatomy of the subject 112 may be estimated using images reconstructed from point cloud data, the point cloud data being generated by the camera image data processor 215 according to depth images received from the depth camera 114. The computing device 216 may use these parameters of the subject to perform, for example, patient-scanner contact prediction, scan range superposition, and scan key point calibration, as will be described in further detail herein. Further, data from the depth camera 114 may be displayed by means of the display 232.

The CT imaging system 100 may perform imaging examination on the basis of a scanning protocol. The scanning protocol is a description of the imaging examination. The scanning protocol may include a description of an involved body part, for example, a medical or colloquial term for the body part. The scanning protocol may provide various parameters and related information for performing scans and post-processing, such as power value, duration of radiation, speed of movement, radiation energy, time delay between image captures, etc. It is conceivable that any configurable technical parameter that should be used for imaging examination by the imaging system 110 may be defined in the scanning protocol.

The CT imaging system 100 may have an automatic patient positioning function. That is, a patient may be automatically positioned in a scan start position in an opening of the gantry 102 on the basis of an examination instruction or the scanning protocol, and moved to a scan end position during scanning and imaging in the scanning direction (e.g., a Z-axis direction in the coordinate system shown in FIG. 1, namely, a direction in which the scanning table 115 and the subject 112 carried on the scanning table enter or exit the opening of the gantry 102). The scan start position and the scan end position may be visually presented using the methods described in the present disclosure, and change as the operator adjusts the scan range indicator.

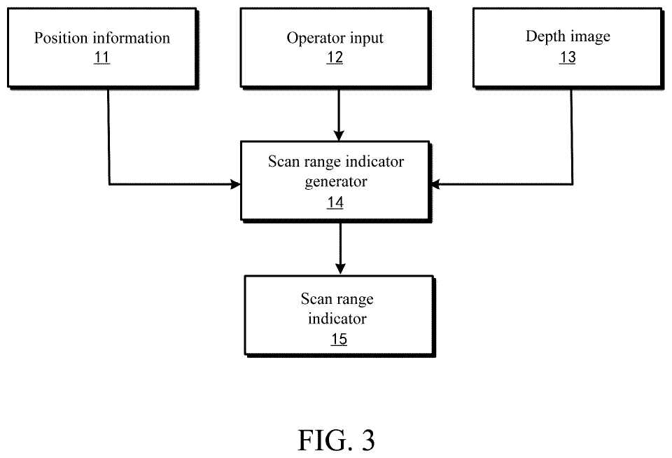

According to embodiments of the present disclosure, a scan range indicator is created on the basis of a depth image of a patient. In order to eliminate the parallax problem, a 3D point cloud of the head region of the patient is filled in. The scan range indicator of the present disclosure is generated on the basis of the filled-in 3D point cloud of the patient. FIG. 3 illustrates a logic diagram of generating a scan range indicator according to a technique of the present disclosure.

The scan range indicator 15 is generated by a scan range indicator generator 14 on the basis of position information 11, an operator input 12 and a depth image 13. The scan range indicator generator 14 may be implemented, for example, as the computing device 216 shown in FIG. 2, the camera image data processor 215, or another separate computing device.

The position information 11 may include position information of the scanning table 115, which may be obtained from, for example, the scanning table motor controller 226 and the gantry motor controller 212. The scanning table 115 includes a head support. The head support is used to affix the patient's head, and the position thereof relative to the scanning table 115 is fixed. Thus, based on the position information 11, it is possible to determine the position of the head support, for example, a position in the coordinate system of the medical imaging system, thereby determining a junction position of a head region and a torso region in an image of the patient.

The operator input 12 may be obtained from an operator console 220. The operator input 12 may include original scan start (upper boundary) and end (lower boundary) positions, as well as real-time adjustment inputs to the scan start (upper boundary) and/or end (lower boundary) positions.

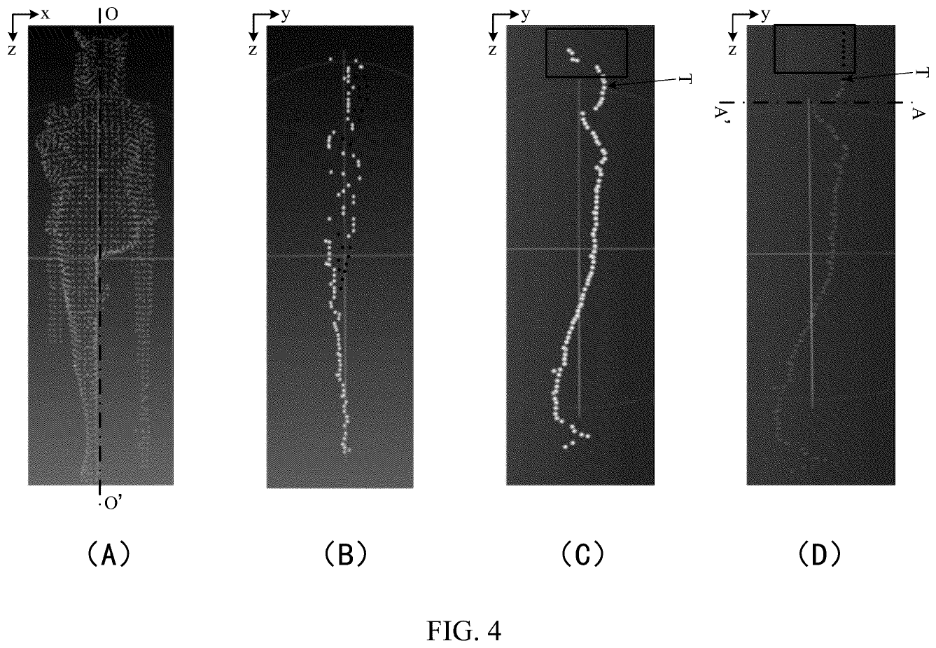

The depth image 13 may be obtained from the depth camera 114. The scan range indicator generator 14 may generate an original 3D point cloud on the basis of the depth image 13. FIGS. 4(A) to 4(D) illustrate schematic diagrams of a process for processing an original 3D point cloud of a patient according to a technique of the present disclosure.

FIG. 4(A) shows the original 3D point cloud generated from the depth image 13. The original 3D point cloud includes three-dimensional point cloud information of an examination subject. The density of the point cloud is determined by the resolution of the depth image and the image processing algorithm used, which is not limited in the present disclosure. In the present embodiment, a 3D point cloud of an entire body of the examination subject is generated. In some embodiments, only a depth image of a portion of the examination subject may be acquired, or only a 3D point cloud of a portion of the examination subject may be generated, which may depend on a desired scan range.

After the original 3D point cloud shown in FIG. 4(A) is generated, the original 3D point cloud is projected onto a plane perpendicular to a scanning table and along a scanning direction (a direction along a line OO′ and perpendicular to a paper surface in FIG. 4(A)) to obtain a projected 2D point cloud, as shown in FIG. 4(B). Specifically, X-axis (refer to FIG. 1) coordinate information of the original 3D point cloud may be unified, and, e.g., set to the X-axis coordinate of the center line of the scanning table (as shown by the line OO′ in FIG. 4(A)), while the Y-axis and Z-axis coordinates of each point are retained. It should be noted that FIG. 4(A) is a view looking down from above the examination subject. FIGS. 4(B) to 4(D) are views of a projection plane (i.e., a plane along the line OO′ and perpendicular to the plane shown in FIG. 4(A)) viewed from a projection direction.

When the original 3D point cloud and the projected 2D point cloud shown in FIGS. 4(A) and 4(B) are generated, filtering processing may be performed on the original 3D point cloud and/or the projected 2D point cloud. The filtering processing may remove redundant points in the image. The redundant points may be caused by an object other than the examination subject, for example a device or another object in a room. The object other than the examination subject (patient) can be identified on the basis of scanning table position in the position information 11, so that the point clouds of these objects are removed.

After the projected 2D point cloud is generated, a patient 2D point cloud contour may further be generated. The projected 2D point cloud may be traversed along the scanning direction (i.e., the Z-axis), a highest point at each Z-axis position selected and other points removed to generate a 2D point cloud contour. In other words, the 2D point cloud contour only retains the highest points at the individual scan positions, so that the generated 2D point cloud contour matches the surface contour of the examination subject. It can be understood that when the height of a middle part such as the lower body of the patient as the examination subject is lower than the heights of the legs on both sides, the 2D point cloud contour corresponding to the middle part is the surface contours of the legs on both sides.

Next, the position of the head support of the scanning table can be determined based on the position information 11, thereby determining the head region and the torso region of the patient as the examination subject. A line AA′ in FIG. 4(D) shows a junction between the head region and the torso region, which can be determined on the basis of the position of the head support. The left side of the line AA′ is the head region and the right side of the line AA′ is the torso region.

The highest point T in the head region may be further identified. In the case of a patient lying flat, the highest point T is typically the position of the tip of the nose of the patient. After the highest point T is identified, the 2D point cloud contour is filled in. Specifically, the heights (Y-axis coordinates) of all points located on one side of the top of the head relative to the highest point T may be replaced with the height (Y-axis coordinate) of the highest point T. As a result, comparing FIG. 4(C) with FIG. 4(D), the point cloud on the left side of the highest point T is leveled to the same height as the highest point T. By means of generating the scan range indicator on the basis of the filled-in 2D point cloud, the parallax problem described above with reference to FIG. 2 can be eliminated.

According to the present disclosure, filling processing on the head region takes into account the general mounting position of the depth camera, i.e., above an area below the patient's head, such that the entire scanning table is in the field of view of the depth camera within the movable range. Therefore, the parallax problem easily occurs when the scan range is set from the top of the head. However, depending on the size and position of a scan subject and the scan range, said filling processing may not be performed. For example, when only a position at the thorax or lower needs to be scanned, the upper boundary position of the scan range may be just below the depth camera, and no parallax problem will be created. In this case, the filling processing described herein need not be performed. Thus, in some embodiments, whether to perform the filling-in processing may be determined on the basis of the upper boundary position and the lower boundary position of the real-time scan range. The filling processing may be enabled only when the upper boundary position and/or the lower boundary position is relatively far from the projection position of the depth camera on the scanning table in the scanning direction.

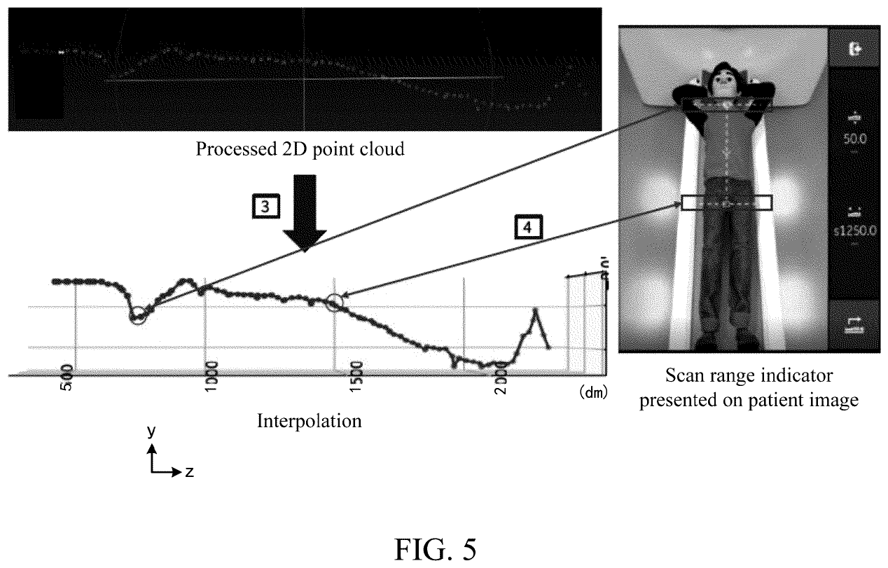

FIG. 5 illustrates a schematic diagram of generating a scan range indicator according to a technique of the present disclosure. The upper left part of FIG. 5 corresponds to the processed 2D point cloud shown in FIG. 4(D), in which the highest point cloud is retained and the head region is filled in. The operator may input a scan start position and a scan end position by inputting a scan plan by means of the operator console 220 or selecting a scan position directly on the image. The scan start position and the scan end position may be represented by coordinates in the scanning direction (i.e., the z-axis).

After the scan start position and the scan end position are acquired, two corresponding points in the 2D point cloud that have the same scan positions as the scan start position and the scan end position may be identified. Since the 2D point cloud is a set of discrete points, there may be no point that completely coincides with the scanning direction coordinates of the scan start position and the scan end position. In this case, points on both sides closest to the scan start position and the scan end position may be interpolated to generate corresponding points. In other words, the points on both sides closest to the scan start position may be interpolated to generate one corresponding point at the scan start position, and the points on both sides closest to the scan end position may be interpolated to generate the other corresponding point at the scan end position, as shown at two circles in the figure. When there is a point corresponding to the scan start position or the scan end position, no interpolation processing is required, and a point in the 2D point cloud at the position may be directly used as the corresponding point.

After the coordinates of the corresponding points are determined, a scan range indicator may be presented on the image of the patient based on the coordinates of the corresponding points. The image of the patient may be an original image obtained from the depth camera. The scan range indicator includes an upper boundary line and a lower boundary line. In the present embodiment, the upper boundary line and the lower boundary line have a linear shape, and points therein correspond to the coordinates of the corresponding points. In other words, the midpoints of the upper boundary line and the lower boundary line may be first determined and then extended by a certain length along the plane of the scanning table to both sides of the patient. The upper boundary line and the lower boundary line may be represented by a dotted line as shown in FIG. 5, or may be represented by a solid line or in another manner.

By employing the filling processing of the 2D point cloud and the linear scan range indicator in the present disclosure, it is possible to perform filling-in processing on the point cloud in a portion having a large angle from the depth camera, such as a top region of the head so that the presented scan range indicator is consistent with the actual scan range.

Further, by employing the embodiments of the present disclosure, even at a scan position where the height (Y-axis) of a central position such as the lower body of the patient as the examination subject is lower than the heights on both sides, the scan range indicator can still be generated on the basis of the highest positions of both sides, so that it can be ensured that the scan range indicator always follows the outer contour of the body of the patient, and in the scanning direction, there are no jumps or distortions in the height direction.

Further, the scan range indicator of the present embodiment is generated on the basis of the corresponding points on the processed 2D point cloud contour of the examination subject. The two corresponding points, once determined, can be used as the midpoints of the upper boundary line and the lower boundary line of the scan range indicator. An upper boundary line and a lower boundary line that have linear shapes can then be generated by extending to both sides.

Further, according to an embodiment of the present disclosure, interpolation processing is performed when the corresponding points in the 2D point cloud contour and corresponding to the upper boundary position and the lower boundary position are determined, so that the actually presented scan range indicator can be continuously moved instead of being intermittently translated with the 2D point cloud contour.

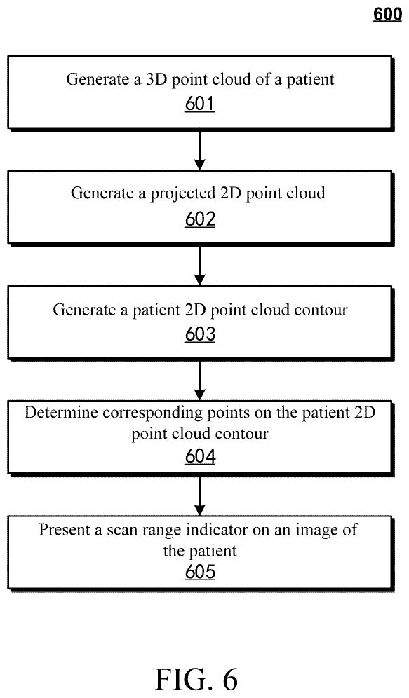

FIG. 6 illustrates a flow diagram of an example generation method 600 of a scan range indicator, according to a technique of the present disclosure.

In step 601, a 3D point cloud of a patient is generated on the basis of a depth image of the patient.

In step 602, the 3D point cloud generated at step 601 is projected onto a plane perpendicular to a scanning table supporting the patient and along a scanning direction (a plane along a line OO′ and perpendicular to an XZ plane in FIG. 4(A)) to generate a projected 2D point cloud. The X-axis (refer to FIG. 1) coordinate information of the original 3D point cloud may be unified, and, e.g., set to the X-axis coordinate of the center line of the scanning table (as shown by the line OO′ in FIG. 4(A)), while the Y-axis and Z-axis coordinates of each point are retained.

In step 603, a patient 2D point cloud contour is generated on the basis of the projected 2D point cloud generated at step 602. The projected 2D point cloud is traversed along the scanning direction (i.e., the Z-axis), the highest point at each Z-axis position is selected and other points are removed, to generate the 2D point cloud contour. In other words, the 2D point cloud contour only retains the highest points at the individual scan positions, so that the generated 2D point cloud contour matches the surface contour of the examination subject.

In step 604, two corresponding points on the patient 2D point cloud contour are determined on the basis of an upper boundary position and a lower boundary position that are received in real time and indicate a scan range, the upper boundary position and the lower boundary position indicating scan positions in the scanning direction. When there are no corresponding points that are completely consistent with the scanning direction coordinates of the upper boundary position and the lower boundary position, the corresponding points may be generated by interpolating points on both sides closest to the upper boundary position and the lower boundary position, respectively. In other words, the points on both sides closest to the upper boundary position may be interpolated to generate one of the corresponding points at the scan start position, and the points on both sides closest to the lower boundary position may be interpolated to generate the other of the corresponding points at the scan end position. In some embodiments, interpolation processing may also not be performed, and only the points on the patient 2D point cloud contour that are closest to the upper boundary position and the lower boundary position are taken as the corresponding points.

In step 605, using the positions of the corresponding points determined at step 604 as base points, an upper boundary line and a lower boundary line of the scan range are presented on the image of the patient as a scan range indicator, as shown in FIG. 5.

In some embodiments, the upper boundary line and the lower boundary line may have a linear shape. A midpoint of the upper boundary line may coincide with one of the corresponding points, and a midpoint of the lower boundary line may coincide with the other of the corresponding points.

In some embodiments, the projected 2D point cloud of the patient may be divided into a head region and a torso region on the basis of the position information of the scanning table, in particular the position information included therein for placing the patient's head. The highest point in the head region is then identified, and the heights of all points on the patient 2D point cloud contour that are located at one side of the top of the patient's head relative to the highest point are replaced with the height of the highest point. It should be understood that the identification of the head region and the torso region is used to implement the filling processing described herein. However, the filling processing may be selectively enabled or disabled depending on a desired scan range. Whether to enable the filling processing may be determined on the basis of the positions of the upper boundary position and the lower boundary position of the scan range indicator relative to the depth camera which are input by the operator. When the upper boundary position and/or the lower boundary position has a relatively large angle of view with respect to the depth camera, i.e., a line connecting the upper boundary position and/or the lower boundary position with the depth camera has a relatively large angle with respect to a vertical line passing through the depth camera, it is determined that parallax is more likely to occur, and thus the filling processing may be enabled.

In addition, the region that is filled in is not limited to the head region described herein, and the region that needs to be filled in may also be divided on the basis of the position of the depth camera. In some embodiments, a range of regions that need not be filled in may be predetermined. The predetermined range of regions that need not be filled in can be determined on the basis of the mounting position and the field of view of the depth camera. When any one of the upper boundary position and the lower boundary position of the scan range indicator input by the operator exceeds the range of regions, an excess portion may be identified as a region that needs to be filled in. In this case, a highest point of the excess portion may be determined and the filling processing may then be performed as described above. Thus, even if the head region of the examination subject is not identified or the examination subject is an object other than a human being, the filling processing of the present disclosure can be implemented, thereby improving the parallax problem.

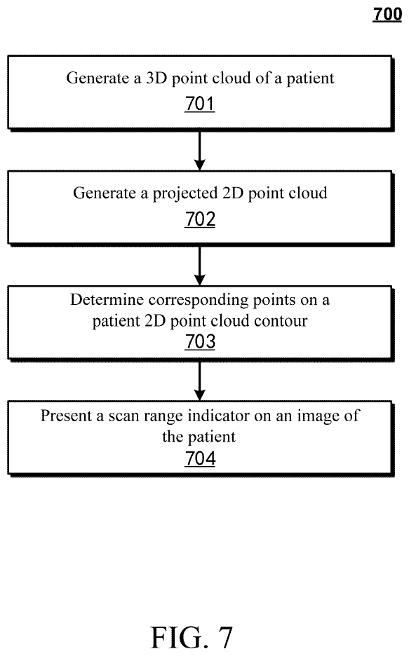

FIG. 7 illustrates a flow diagram of another example generation method 700 of a scan range indicator according to a technique of the present disclosure. The present embodiment, in contrast to the method 600 shown in FIG. 6, does not generate a corresponding 2D point cloud contour, but rather generates points corresponding to an upper boundary position and a lower boundary position of the scan range indicator directly on a projected 2D point cloud.

In particular, in step 701, a 3D point cloud of a patient is generated based on a depth image of the patient.

In step 702, the 3D point cloud generated at step 701 is projected onto a plane perpendicular to a scanning table supporting the patient and along a scanning direction to generate a projected 2D point cloud.

In step 703, corresponding points in the projected 2D point cloud are determined on the basis of an upper boundary position and a lower boundary position that are received in real time and indicate a scan range, the upper boundary position and the lower boundary position indicating scan positions in the scanning direction, and the corresponding points being highest points at the corresponding scan positions. In this step, after the coordinates of the upper boundary position and the lower boundary position are acquired, a point cloud array having corresponding scanning axial coordinates in the projected 2D point cloud may be determined, and a highest point in the corresponding point cloud array, namely, a point having the largest Y-axis coordinate may be used as the corresponding point. Compared with the method 600, the present embodiment can obtain good continuity without interpolation processing, and since point cloud information other than the 2D point cloud contour is retained, points closer to the scanning axial coordinates of the upper boundary position and the lower boundary position can be found as the corresponding points for generating a scan range indicator.

In step 704, using the scan positions (namely, positions in the scanning direction or positions on the Z-axis) of the corresponding points determined in step 703 as base points, an upper boundary line and a lower boundary line of the scan range are presented on the image of the patient as a scan range indicator.

In some embodiments, the upper boundary line and the lower boundary line may have a linear shape. A midpoint of the upper boundary line may coincide with one of the corresponding points, and a midpoint of the lower boundary line may coincide with the other of the corresponding points.

In some embodiments, the projected 2D point cloud of the patient may be divided into a head region and a torso region on the basis of position information of the scanning table. The highest point in the head region is then identified, and the heights of all points in the patient 2D point cloud that are located on one side of the top of the patient's head relative to the highest point are replaced with the height of the highest point. In this way, even without generating 2D point cloud contour as shown in FIG. 4(C) in advance, the filled-in 2D point cloud contour as shown in FIG. 4(D) can still be obtained.

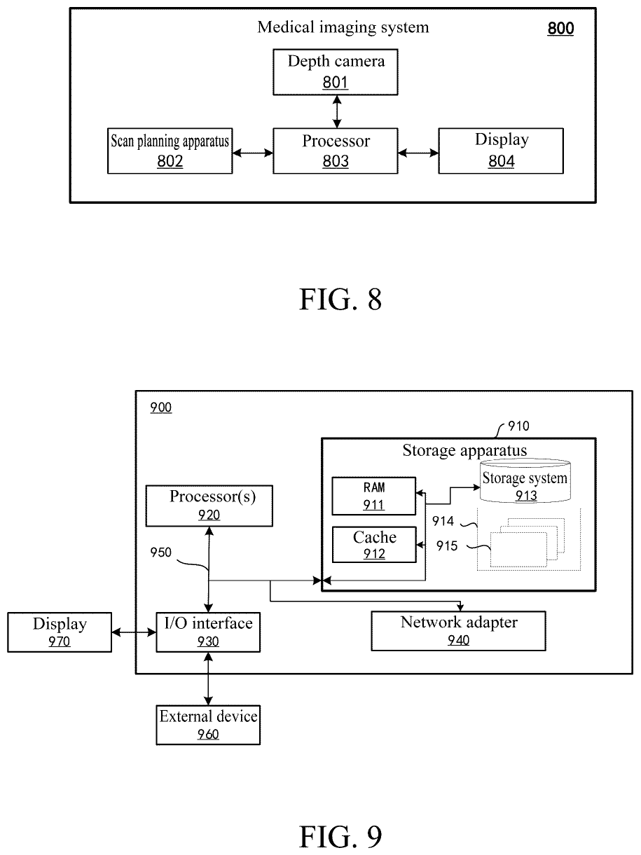

FIG. 8 illustrates a block diagram of an exemplary imaging system 800 according to a technique of the present disclosure. In various implementations, the medical imaging system 800 may include the imaging system 200 shown in FIG. 2, and used to implement the example logic shown in FIG. 3. The medical imaging system 800 generally includes a depth camera 801, a scan planning apparatus 802, a processor 803, and a display 804. The depth camera 801 is fixedly mounted in a room in which the system 800 is located, as described above, and is configured to acquire a depth image of a scan subject supported on a scanning table. The scan planning apparatus 802 may refer to the operator console 220 shown in FIG. 2, and is used to receive an upper boundary position and a lower boundary position that are input by an operator and indicate a scan range, and to provide the upper boundary position and the lower boundary position to the processor 803. The processor 803 is used to process the depth image acquired by the depth camera 801 by means of the methods 600, 700 described in the present disclosure, and to cause the display 804 to present the upper boundary position and the lower boundary position of the scan range in the manner described in the present disclosure on the basis of the upper boundary position and the lower boundary position received from the scan planning apparatus 802.

FIG. 9 illustrates an exemplary block diagram of a computing device 900 according to a technique of the present disclosure. The computing device 900 may be implemented as an example of the computing device 216 shown in FIG. 2. The computing device 900 includes: one or a plurality of processors 920; and a storage apparatus 910, used to store one or a plurality of programs, the one or plurality of programs, when executed by the one or plurality of processors 920, causing the one or plurality of processors 920 to implement the procedures described in the present disclosure. The processor is, for example, a digital signal processor (DSP), a microcontroller, an application-specific integrated circuit (ASIC), or a microprocessor.

The computing apparatus 900 shown in FIG. 9 is merely an example, and should not cause any limitation to the function and use scope of the embodiments of the present disclosure.

As shown in FIG. 9, the computing device 900 is represented in the form of a general-purpose computing device. Assemblies of the computing device 900 may include, but are not limited to: one or a plurality of processors 920, a storage apparatus 910, and a bus 950 connecting different system components (including the storage apparatus 910 and the processor 920).

The bus 950 indicates one or a plurality of types among several types of bus structures, including a memory bus or memory controller, a peripheral bus, a graphics acceleration port, a processor, or a local bus using any bus structure of the plurality of bus structures. For example, these architectures include, but are not limited to, an industry standard architecture (ISA) bus, a microchannel architecture (MCA) bus, an enhanced ISA bus, a video electronics standards association (VESA) local bus, and a peripheral component interconnection (PCI) bus.

The computing device 900 typically includes multiple types of computer system readable media. These media may be any available medium that can be accessed by the computing device 900, including volatile and non-volatile media as well as removable and non-removable media.

The storage apparatus 910 may include a computer system readable medium in the form of a volatile memory, for example, a random access memory (RAM) 911 and/or a cache memory 912. The computing device 900 may further include other removable/non-removable, and volatile/non-volatile computer system storage media. For example only, the storage system 913 may be configured to read and write a non-removable, non-volatile magnetic medium (which is not shown in FIG. 9, and is generally referred to as a “hard drive”). Although not shown in FIG. 9, a magnetic disk drive for reading and writing a removable non-volatile magnetic disk (such as a “floppy disk”) and an optical disc drive for reading and writing a removable non-volatile optical disc (such as a CD-ROM, a DVD-ROM, or other optical media) may be provided. In these cases, each drive may be connected to the bus 950 by means of one or a plurality of data medium interfaces. The storage device 910 may include at least one program product which has a group of program modules (for example, at least one program module) configured to execute the functions of the embodiments of the present disclosure.

A program/utility tool 914 having a group (at least one) of program modules 915 may be stored in, for example, the storage apparatus 910. This program module 915 includes, but is not limited to, an operating system, one or a plurality of application programs, other program modules, and program data, and each of these examples or a certain combination thereof may include implementation of a network environment. The program module 915 typically executes the function and/or method in any embodiment described in the present disclosure.

The computing device 900 may also communicate with one or a plurality of peripheral devices 960 (such as a keyboard, a pointing device, and a display 970), and may also communicate with one or a plurality of devices that enable a user to interact with the computing device 900, and/or communicate with any device (such as a network card and a modem) that enables the computing device 900 to communicate with one or a plurality of other computing devices. Such communication may be performed via an input/output (I/O) interface 930. Moreover, the computing device 900 may also communicate with one or a plurality of networks (for example, a local area network (LAN), a wide area network (WAN) and/or a public network, for example, the Internet) through a network adapter 940. As shown in FIG. 9, the network adapter 940 communicates with other modules of the computing device 900 through the bus 950. It should be understood that although not shown in the figure, other hardware and/or software modules can be used in combination with the computing device 900, including, but not limited to, microcode, device drivers, redundant processing units, external disk drive arrays, RAID systems, tape drives, data backup storage systems, and the like.

The processor 920 executes various functional applications and data processing, for example implementing the procedures described in the present disclosure, by running programs stored in the storage apparatus 910.

The technique described herein may be implemented with hardware, software, firmware, or any combination thereof, unless specifically described as being implemented in a specific manner. Any features described as modules or components may also be implemented together in an integrated logical apparatus, or separately implemented as discrete but interoperable logical apparatuses. If implemented with software, the technique may be implemented at least in part by a non-transitory processor-readable storage medium that includes instructions, where when executed, the instructions perform one or more of the aforementioned methods. The non-transitory processor-readable data storage medium may form part of a computer program product that may include an encapsulation material. Program code may be implemented in a high-level procedural programming language or an object-oriented programming language so as to communicate with a processing system. If desired, the program code may also be implemented in an assembly language or a machine language. In fact, the mechanisms described herein are not limited to the scope of any particular programming language. In any case, the language may be a compiled language or an interpreted language.

One or a plurality of aspects of at least some embodiments may be implemented by representative instructions that are stored in a machine-readable medium and represent various logic in a processor, where when read by a machine, the representative instructions cause the machine to manufacture the logic for executing the technique described herein.

Such machine-readable storage media may include, but are not limited to, a non-transitory tangible arrangement of an article manufactured or formed by a machine or device, including storage media, such as: a hard disk; any other types of disk, including a floppy disk, an optical disk, a compact disk read-only memory (CD-ROM), compact disk rewritable (CD-RW), and a magneto-optical disk; a semiconductor device such as a read-only memory (ROM), a random access memory (RAM) such as a dynamic random access memory (DRAM) and a static random access memory (SRAM), an erasable programmable read-only memory (EPROM), a flash memory, and an electrically erasable programmable read-only memory (EEPROM); a phase change memory (PCM); a magnetic or optical card; or any other type of medium suitable for storing electronic instructions.

Instructions may further be sent or received by means of a network interface device that uses any of a number of transport protocols (for example, Frame Relay, Internet Protocol (IP), Transfer Control Protocol (TCP), User Datagram Protocol (UDP), and Hypertext Transfer Protocol (HTTP)) and through a communication network using a transmission medium.

An exemplary communication network may include a local area network (LAN), a wide area network (WAN), a packet data network (for example, the Internet), a mobile phone network (for example, a cellular network), a plain old telephone service (POTS) network, and a wireless data network (for example, Institute of Electrical and Electronics Engineers (IEEE) 802.11 standards referred to as Wi-Fi®, and IEEE 802.16 standards referred to as WiMax®), IEEE 802.15.4 standards, a peer-to-peer (P2P) network, and the like. In one example, the network interface device may include one or a plurality of physical jacks (for example, Ethernet, coaxial, or phone jacks) or one or a plurality of antennas for connection to the communication network. In one example, the network interface device may include a plurality of antennas that wirelessly communicate using at least one of single-input multiple-output (SIMO), multiple-input multiple-output (MIMO), and multiple-input single-output (MISO) technology.

The term “transmission medium” should be considered to include any intangible medium capable of storing, encoding, or carrying instructions for execution by a machine, and the “transmission medium” includes digital or analog communication signals or any other intangible medium for facilitating communication of such software.

Some exemplary embodiments have been described above. However, it should be understood that various modifications can be made to the exemplary embodiments described above without departing from the spirit and scope of the present disclosure. For example, an appropriate result can be achieved if the described techniques are performed in a different order and/or if the components of the described system, architecture, apparatus, or circuit are combined in other manners and/or replaced or supplemented with additional components or equivalents thereof; accordingly, the modified other embodiments also fall within the protection scope of the claims.

1. A method for generating a scan range indicator in a medical imaging system, comprising:
projecting a 3D point cloud generated on the basis of a depth image of a patient onto a plane perpendicular to a scanning table supporting the patient and along a scanning direction, so as to generate a projected 2D point cloud;
generating a patient 2D point cloud contour on the basis of the projected 2D point cloud;
on the basis of an upper boundary position and a lower boundary position that are received in real time and indicate a scan range, determining two corresponding points on the patient 2D point cloud contour, the upper boundary position and the lower boundary position indicating scan positions in the scanning direction; and
using the scan positions of the corresponding points as base points, presenting an upper boundary line and a lower boundary line of the scan range on an image of the patient as a scan range indicator.

2. The method according to claim 1, wherein the upper boundary line and the lower boundary line have a linear shape.

3. The method according to claim 2, wherein a midpoint of the upper boundary line coincides with one of the corresponding points, and a midpoint of the lower boundary line coincides with the other of the corresponding points.

4. The method according to claim 1, wherein the patient 2D point cloud contour is generated by means of selecting a highest point at each scan position as a contour height.

5. The method according to claim 1, further including:
dividing the projected 2D point cloud of the patient into a head region and a torso region on the basis of position information of the scanning table;
identifying a highest point in the head region; and
replacing, with the height of the highest point, the heights of all points on the patient 2D point cloud contour that are located at one side of the top of the patient's head relative to the highest point.

6. The method according to claim 5, wherein the position information of the scanning table includes position information of a support for placing the patient's head.

7. The method according to claim 1, wherein determining the two corresponding points on the patient 2D point cloud contour includes: determining points on the patient 2D point cloud contour that are closest to the upper boundary position and the lower boundary position, respectively, to be the corresponding points.

8. The method according to claim 1, wherein determining the two corresponding points on the patient 2D point cloud contour includes interpolating nearest points on both sides of the upper boundary position and the lower boundary position, respectively, to be the corresponding points.

9. The method according to claim 1, wherein the depth image of the patient is obtained by means of a depth camera which is fixedly mounted in the medical imaging system.

10. A method for generating a scan range indicator in a medical imaging system, comprising:
projecting a 3D point cloud, generated on the basis of a depth image of a patient, onto a plane perpendicular to a scanning table supporting the patient and along a scanning direction, so as to generate a projected 2D point cloud;
on the basis of an upper boundary position and a lower boundary position that are received in real time and indicate a scan range, determining corresponding points in the projected 2D point cloud, the upper boundary position and the lower boundary position indicating scan positions in the scanning direction, and the corresponding points being highest points at corresponding scan positions thereof; and
using the scan positions of the corresponding points as base points, presenting an upper boundary line and a lower boundary line of the scan range on an image of the patient as a scan range indicator.

11. The method according to claim 10, wherein the upper boundary line and the lower boundary line have a linear shape.

12. The method according to claim 11, wherein a midpoint of the upper boundary line coincides with one of the corresponding points, and a midpoint of the lower boundary line coincides with the other of the corresponding points.

13. The method according to claim 10, further including:
dividing the projected 2D point cloud of the patient into a head region and a torso region on the basis of position information of the scanning table;
identifying a highest point in the head region; and
replacing, with the height of the highest point, the heights of all points in the patient 2D point cloud contour that are located at one side of the top of the patient's head relative to the highest point.

14. The method according to claim 13, wherein the position information of the scanning table includes position information of a support for placing the patient's head.

15. The method according to claim 10, wherein determining the corresponding points in the projected 2D point cloud includes: determining highest points at the scan positions closest to the upper boundary position and the lower boundary position on the patient 2D point cloud contour, respectively, to be the corresponding points.

16. The method according to claim 10, wherein determining the corresponding points in the projected 2D point cloud includes: interpolating highest points at the scan positions on both sides of the upper boundary position and the lower boundary position, respectively, to be the corresponding points.

17. The method according to claim 10, wherein the depth image of the patient is obtained by means of a depth camera which is fixedly mounted in the medical imaging system.

18. A medical imaging system, comprising:
a depth camera configured to acquire a depth image of a patient;
a scanning table configured to support the patient;
a scan planning apparatus configured to receive an upper boundary position and a lower boundary position that indicate a scan range;
a display configured to display an image of the patient; and
a processor, configured to:
project a 3D point cloud, generated on the basis of the depth image of the patient acquired by the depth camera, onto a plane perpendicular to the scanning table and along a scanning direction, so as to generate a projected 2D point cloud;
generate a patient 2D point cloud contour on the basis of the projected 2D point cloud;
on the basis of the upper boundary position and the lower boundary position that are received in real time and indicate the scan range, determine corresponding points on the patient 2D point cloud contour, the upper boundary position and the lower boundary position indicating scan positions in the scanning direction; and
using the scan positions of the corresponding points as base points, cause the display to present an upper boundary line and a lower boundary line of the scan range on an image of the patient.
